(19) United States
(12) Patent Application Publication
(10) Pub. No.: US 2021/0196913 A1
(43) Pub. Date: Jul. 1, 2021

(54) VENTILATOR AND PROCESS FOR OPERATING A VENTILATOR

(71) Applicant: Drägerwerk AG & Co. KGaA, Lübeck (DE)

(72) Inventor: Hans-Ullrich HANSMANN, Barnitz (DE)

(21) Appl. No.: 17/057,785

(22) PCT Filed: Apr. 9, 2019
(86) PCT No.: PCT/EP2019/058877

§ 371 Date: Nov. 23, 2020

(30) Foreign Application Priority Data
May 31, 2018 (DE) 10 2018 004 341.0

(51) Int. Cl. A61M 16/00; A61M 16/20

(52) U.S. Cl. CPC A61M 16/024; A61M 16/0057; A61M 16/208; A61M 2016/0027; A61M 16/205; A61M 2205/0294; A61M 16/204

(57) ABSTRACT

A respiratory device (10) includes at least one expiration valve (12) and/or at least one inspiration valve (14) with a valve drive (18) that is configured to influence a position of a closure element (20) of each valve (12, 14). A method for operating such a respiratory device (10) is also provided. The valve drive (18) acts on a valve chamber (24) and a volume in the valve chamber (24) determines the position of the closure element (20). The valve drive (18) includes a plurality of piezo pumps (40, 42), with at least one regular piezo pump (40) with a direction of action towards the valve chamber (24) and at least one inverse piezo pump (42) with a reverse direction of action.

### CROSS REFERENCE TO RELATED APPLICATIONS

This application is a United States National Phase Application of International Application PCT/EP2019/058877, filed Apr. 9, 2019, and claims the benefit of priority under 35 U.S.C. § 119 of German Application 10 2018 004 341.0, filed May 31, 2018, the entire contents of which are incorporated herein by reference.

### TECHNICAL FIELD

The present invention pertains to a ventilator and to a process for operating a ventilator, especially to a mobile ventilator worn by the patient, but basically likewise to a ventilator in the form of a ventilator suitable for clinical use, especially in the form of a combined anesthesia device and ventilator. The present invention further pertains to a process for operating such a ventilator.

### TECHNICAL BACKGROUND

Ventilators are well known per se. A ventilator or a ventilator in the form of a combined anesthesia device and ventilator, designated below in summary as ventilator, functions in a manner known per se as a breathing gas supply unit, for example, by the ventilator being connected to an external gas supply or itself comprising a breathing gas supply unit, for example, in the form of a pump, of a fan impeller or the like. The pressure on the side of the ventilator is raised by means of the ventilator in a manner that is likewise known per se to a predefined or predefinable desired value for the airway pressure during inhalation, namely a value above the so-called alveolar pressure, i.e., the pressure within a patient's lungs. This pressure difference leads to a volume flow in the direction towards the patient's lungs. The volume flow disappears when pressure equalization is reached. The process is reversed during exhalation and the pressure on the side of the ventilator is lowered compared to the alveolar pressure, so that a volume flow arises from the patient's lungs until a pressure equalization has taken place here as well.

A pressure control, a volume control and various mixed forms with different limitations are known for such an operation of a ventilator. During the operation of the ventilator, valves on the input side and on the output side (inhalation valve, exhalation valve) are actuated and are opened or closed in a defined manner in a manner basically known per se.

In prior-art ventilators, a relief of the exhalation valve takes place at the beginning of the expiratory phase due to a dynamic pressure developing during exhalation of the patient and acting on a closing body of the exhalation valve. The patient must thus at least briefly “breathe against the exhalation valve,” so that this exhalation valve opens. This is perceived by the patient in some cases and then possibly found to be disturbing.

### SUMMARY

Based on this background, an object of the present invention is to provide a ventilator with at least one valve comprised by it with improved dynamic properties, especially to provide a ventilator, in which the perceptions that are mentioned above and are uncomfortable for a patient are avoided.

The innovation proposed here is a ventilator, which comprises at least one exhalation valve and/or at least one inhalation valve with a special valve drive, namely a valve drive, which comprises at least one pumping device designated here and below as a piezo pump, wherein the valve drive and thus the piezo pump or each piezo pump comprised by it is intended for influencing a position of a closing body of the respective valve. To influence the position of the closing body, the valve drive acts on a valve chamber of the valve by displacing a fluid into the valve chamber or out of the valve chamber and a volume in the valve chamber determines the position of the closing body. The fluid that is displaced into the valve chamber or out of the valve chamber by means of the valve drive is preferably a gas, especially ambient air. The special feature of the ventilator is that the valve drive comprises a plurality of piezo pumps, namely at least one piezo pump, regular piezo pump, with a direction of action for displacing a fluid towards the valve chamber and at least partially into the valve chamber as well as at least one piezo pump, inverse piezo pump, with a direction of action for displacing the fluid away from the valve chamber and at least partially out of the valve chamber.

The direction of action of a piezo pump arises due to a volume flow resulting during an activation of the piezo pump, i.e., a volume flow of the respective fluid generated by means of the piezo pump. In case of a piezo pump with a direction of action towards the valve chamber (regular piezo pump), a volume flow arises in the direction towards the valve chamber and at least partially into the valve chamber during the activation thereof. In case of a piezo pump with a direction of action away from the valve chamber (inverse piezo pump), a volume flow in an opposite direction, i.e., in a direction away from the valve chamber arises during the activation thereof.

By means of the regular piezo pump or each regular piezo pump, the volume in the valve chamber can be increased and the respective valve closes with such an increase in volume. In this case, the pressure in the valve chamber (together with a surface of the closing body, which surface is facing towards the valve chamber) determines the “locking force” of the valve. In the case of a valve drive with a regular piezo pump or with a plurality of regular piezo pumps, this/these is/are deactivated for opening the valve. However, the valve does not necessarily open at the time of deactivation of the valve drive. Rather, a dynamic pressure acting on the closing body and a certain volume flow through the valve after an initial opening of the valve are usually necessary for opening the valve. In case of a valve functioning as exhalation valve, the patient applies this dynamic pressure during exhalation and the volume flow in the form of exhaled breathing air holds the exhalation valve open after the initial opening of the exhalation valve. The opening of the valve by a dynamic pressure being applied “on the outside” is designated below as passive (not caused by the valve drive itself) opening and leads to a significant flow resistance of the valves. An active opening of the valve is possible by means of at least one inverse piezo pump, i.e., an opening caused by the valve drive itself. In case of an activation of the at least one inverse piezo pump comprised by the valve drive, the volume in the valve chamber is reduced due to the displacement of fluid away from the valve chamber and at least partially out of the valve chamber, which displacement is triggered by means of the inverse piezo pump, so that a corresponding position change of the closing body of the valve results: The valve opens (is actively opened by means of the at least one inverse piezo pump).

The ventilator being proposed here accomplishes the above-mentioned object because a valve that can be actively opened by means of at least one inverse piezo pump is characterized by improved dynamic properties since the opening can take place very rapidly and the closing body is able to be actively displaced into positions, for which a flow through the valve would otherwise be necessary. The valve may thus be opened at the maximum, for example, even in case of an only minimal flow through the valve, so that a correspondingly reduced flow resistance results. The valve may even be opened independently of a flow through the valve.

The now existing possibility of an active opening of a valve with a miniaturized valve drive, i.e., with a valve drive comprising piezo pumps, allows an opening of the valve, especially an opening of a valve functioning as exhalation valve, independently of a dynamic pressure being applied. In case of a valve functioning as an exhalation valve, this valve can consequently be actively opened immediately at the beginning of the expiratory phase and offers in the opened state a negligibly low flow resistance compared to a passively opening valve.

In case of an actively opening valve functioning as exhalation valve, the ventilated patient is able to exhale a large volume during the expiratory phase even at the beginning thereof without noticing a resistance to be overcome in the process. In addition, a dynamics not hitherto known previously to this extent in case of the control of a volume flow, i.e., in case of blocking or controlling the flow of the volume flow, is possible due to the valve functioning in case of an actively opening valve as an exhalation valve or inhalation valve. This is especially advantageous for transient processes during a transition from an inspiratory phase to a subsequent expiratory phase as well as during a transition from an expiratory phase to a subsequent inspiratory phase during a ventilation of a patient.

The above-mentioned object is also accomplished by means of a process for operating a ventilator of the type described here and below. Provisions are made in such an operating process for the valve that comprises the inverse piezo pump to be actively opened by means of at least one inverse piezo pump. This active opening can take place in case of the valve or each valve comprised by the ventilator and functioning as exhalation valve and/or in case of the valve or each valve comprised by the ventilator and functioning as inhalation valve. In case of a valve functioning as exhalation valve with at least one inverse piezo pump, the exhalation valve is actively opened by means of this at least one inverse piezo pump.

In summary, significant advantages of the innovation being proposed here arise especially because of the possible, controlled or regulated reduction of the flow resistance of the respective valve, i.e., because of the possibility of an active reduction of the flow resistance. This is true especially in the case of a valve functioning as exhalation valve. The now present possibility of the active reduction of the flow resistance of a valve makes possible an increase in the dynamics with regard to transient changes in relieving pressure.

The innovation is especially considered for use because of the miniaturized valve drive in case of a mobile ventilator worn by the patient to be ventilated or generally directly assigned in space to the patient. For example, the use in a ventilator, which is coupled directly to a patient interface worn by the patient, for example, a breathing mask or the like, comes into consideration.

The innovation is not only a ventilator with at least one valve with a valve drive comprising at least one inverse piezo pump, but also such a valve itself, i.e., a valve, especially a pneumatic valve, with a valve drive comprising at least one inverse piezo pump as described here and below.

Advantageous embodiments of the present invention are the subject of the subclaims. References used here refer to the further configuration of the subject of the principal claim by the features of the respective subclaim and shall not be considered to represent abandonment of the wish to achieve an independent, concrete protection for the combinations of features of the dependent subclaims. If the subclaims comprise features which perfect the concrete ventilator in the form of a concretization of a valve comprised by it or in the form of a concretization of a valve drive of such a valve, it is true that the features in question are thus also disclosed independently of the use of the respective valve as a basis for a possible concretization of the valve itself to avoid unnecessary repetitions. It shall be assumed, moreover, in respect to an interpretation of the claims as well as of the description in the case of a more specific concretization of a feature in a dependent claim that such a limitation is not present in the respective preceding claims as well as in a more general embodiment of the concrete ventilator/valve. Any reference in the description to aspects of dependent claims shall accordingly also expressly imply a description of optional features even without a special reference. Finally, it should be noted that the ventilator proposed here can also be perfected corresponding to the dependent process claims and vice versa. A variant of the ventilator corresponding to dependent process claims is characterized, for example, in that the ventilator comprises means for carrying out the respective process step or respective process steps. Provisions are made in an embodiment of the ventilator for the valve drive to comprise the at least one piezo pump with a direction of action towards the valve chamber as well as the at least one piezo pump with a direction of action away from the valve chamber in a series arrangement.

Provisions are made in another embodiment of the ventilator for the valve drive to comprise the at least one piezo pump with a direction of action towards the valve chamber as well as the at least one piezo pump with a direction of action away from the valve chamber in a parallel arrangement.

The configurations of the piezo pumps in series arrangement and/or parallel arrangement in the valve drive make it possible to be able to readily adapt the valve drive structurally to the (usually mostly confined) space conditions of a mobile ventilator.

Provisions are made in a preferred embodiment of the ventilator for the valve drive of a valve functioning as exhalation valve or as inhalation valve to comprise exactly one inverse piezo pump and a plurality of regular piezo pumps. The exactly one inverse piezo pump is sufficient for the active opening of the respective valve and possibly a rapid active opening. The plurality of regular piezo pumps generate in the valve chamber a sufficient control pressure to keep the valve closed even against a high dynamic pressure.

Provisions are made in another preferred embodiment of the ventilator for a nonreturn valve to be arranged in the valve drive at the at least one piezo pump with the direction of action towards the valve chamber such that no escape of the volume from the valve chamber against the direction of action is made possible.

Provisions are made in another preferred embodiment of the ventilator for a nonreturn valve to be arranged in the valve drive at the at least one piezo pump with a direction of action away from the valve chamber such that no escape of the volume from the valve chamber against the direction of action is made possible.

Especially the use of nonreturn valves in parallel arrangements of piezo pumps in the valve drive offers the advantageous possibility that such piezo pumps may also be used, through which flow is at least partially possible without an active actuation, i.e., which have no endlessly high flow resistance. The arrangement of nonreturn valves makes possible in such cases that the valve drive overall is then in a defined state even without active actuation of piezo pumps. Defined and clear states of the components are advantageous for the implementation of as simple as possible but reliable control and regulation concepts for operating the ventilator equipped with piezo pumps, without monitoring the possible operating states by means of any additional sensor mechanisms (pressure flow), partially also in a redundant configuration, since such monitoring is then not necessary.

In an embodiment of the process for operating a ventilator of the type described here and below, provisions are made for a measured pressure value to be detected by means of a pressure sensor assigned in space to a valve of the ventilator (as an exhalation valve or inhalation valve) and for a position of the closing body of the respective valve to be regulated by means of the measured pressure value as actual value and by means of a predefined or predefinable pressure value as desired value. Not only an opening or a closing or a partial opening or a partial closing of the valve, but rather a regulated positioning of the closing body will then takes place due to an actuation of the valve drive of the respective valve to obtain a respectively desired pressure situation, for example, to comply with a positive end expiratory pressure (PEEP).

Provisions are made in a special embodiment of the process for the exhalation valve to be actively opened at the beginning of an expiratory phase, so that the patient can exhale a large quantity of breathing gas at the beginning of the expiratory phase. The duration, during which the exhalation valve is actively opened at the beginning of the expiratory phase, is preferably determined here by a predefined or predefinable duration or depends on a measured value collected during the expiratory phase. In case of a duration dependent on a measured value, during which the exhalation valve is actively opened at the beginning of the expiratory phase, provisions are made for the exhalation valve to be actively opened at the beginning of the expiratory phase and to remain opened until a measured pressure value falls below a predefined or predefinable threshold value. The measured pressure value is detected by means of a pressure sensor assigned in space to the exhalation valve and the measured pressure value codes an airway pressure pAW. To this end, the pressure sensor is arranged upstream of the exhalation valve, at least upstream of the closing body of the exhalation valve in the direction of the volume flow flowing out over the exhalation valve during exhalation.

An automatic actuation of the valve drives of the valves comprised by the ventilator, namely an automatic actuation of the piezo pumps (regular and inverse piezo pumps) comprised by the valve drives each, takes place by means of a control device intended for this purpose. The control device comprises a processing unit in the form of or like a microprocessor in a manner basically known per se as well as a memory, into which is loaded a computer program which can be executed by means of the processing unit and which functions as a control program. The control program is run during the operation of the ventilator. The present invention is thus also a computer program with program code instructions that can be executed by a computer, on the one hand, and a storage medium with such a computer program, i.e., a computer program product with program code means, as well as finally also a control device or a ventilator, in the memory of which such a computer program is loaded or can be loaded as means for carrying out the process and its embodiments, on the other hand.

An exemplary embodiment of the present invention will be explained in more detail below on the basis of the drawings. Objects or components corresponding to one another are provided with the same reference numbers in all figures.

The exemplary embodiment or each exemplary embodiment shall not be considered to represent a limitation of the present invention. Rather, variations and modifications, especially such variants and combinations which the person skilled in the art can find in respect to accomplishing the object, for example, by a combination or variation of individual features contained in the general or special text of the description as well as in the claims and/or in the drawings and lead to a new subject by combinable features, are possible within the scope of the present disclosure. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a ventilator with an exhalation valve and with an inhalation valve;

FIG. 2a is a cross-sectional view showing a piezo pump;

FIG. 2b is a cross-sectional view showing the piezo pump;

FIG. 3 is a schematic view of a piezo pump;

FIG. 4a is a schematic view showing a valve drive of a valve device functioning as exhalation valve or as inhalation valve with a plurality of piezo pumps each shown symbolically according to FIG. 3;

FIG. 4b is a schematic view showing a valve drive of a valve device functioning as exhalation valve or as inhalation valve with a plurality of piezo pumps each shown symbolically according to FIG. 3;

FIG. 5a is a schematic view showing the ventilator according to FIG. 1 with a snapshot of the positions of the inhalation valve and the exhalation valve during inhalation in case of a ventilation of a patient;

FIG. 5b is a schematic view showing the ventilator according to FIG. 1 with a snapshot of the positions of the inhalation valve and the exhalation valve during exhalation in case of a ventilation of a patient;

FIG. 6 is a schematic view showing a valve drive as in FIG. 4 with control units for actuation of the piezo pumps comprised by the valve drive;

FIG. 7 is a time curve of the inhalation and exhalation in case of a ventilation of a patient as well as individual pressure values as desired values for the piezo pumps comprised by the valve drive of the exhalation valve;

FIG. 8 is the time curve as in FIG. 7 with a volume flow curve resulting during the expiratory phase because of the actuation of the piezo pumps comprised by the valve drive of the exhalation valve;

FIG. 9 is a schematic view showing the exhalation valve of the ventilator according to FIG. 1 with a pressure sensor assigned in space to the exhalation valve;

FIG. 10 is a graph showing families of characteristics;

FIG. 11 is a graph showing families of characteristics;

FIG. 12 is a schematic view showing the inhalation valve of the ventilator according to FIG. 1 with a pressure sensor assigned in space to the inhalation valve; and

FIG. 13 is a time curve of the inhalation and exhalation in case of a ventilation of a patient in the form of a time curve of the airway pressure and of the volume flow resulting because of the actuation of the valve drives of the inhalation valve and the exhalation valve.

### DESCRIPTION OF PREFERRED EMBODIMENTS

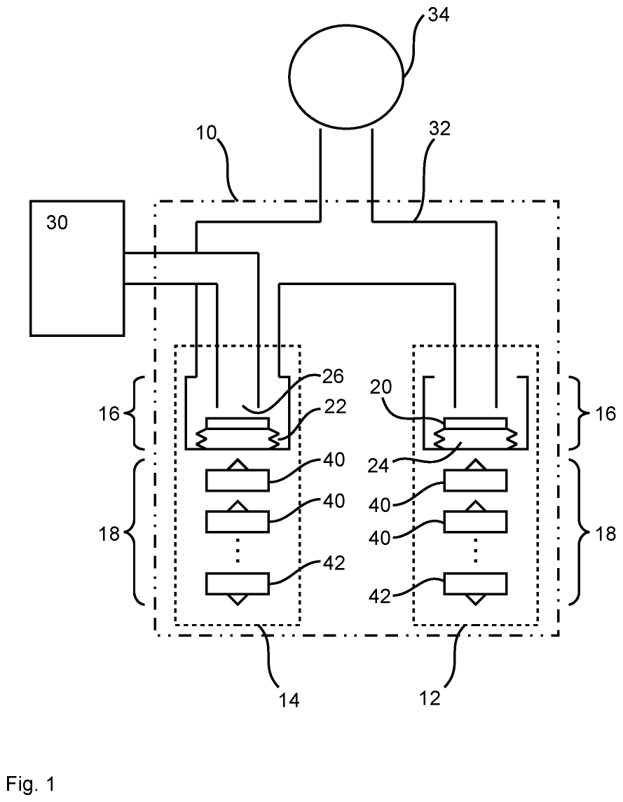

Referring to the drawings, the view in FIG. 1 shows in a schematically very highly simplified general view a ventilator 10 that comprises a first valve device 12 functioning as exhalation valve 12 and a second valve device 14 functioning as inhalation valve 14. The inhalation valve 14 is basically optional. In general, a ventilator 10 according to FIG. 1 comprises at least one exhalation valve 12, i.e., one exhalation valve 12 or possibly a plurality of exhalation valves 12, and an inhalation valve 14 or possibly a plurality of inhalation valves 14 in case of an embodiment with an inhalation valve 14. The further description is continued in the example of a ventilator 10 with exactly one exhalation valve 12 and exactly one inhalation valve 14. More than one exhalation valve and/or inhalation valve 12, 14 each can be implied here. Just the same, it is to be taken into consideration that the inhalation valve 14 is basically optional.

A valve housing 16 and a valve drive 18 belong to each valve device 12, 14 that is comprised by the ventilator 10. A closing body 20, for example, a disk-shaped closing body 20 (valve plate), can be moved by means of the valve drive 18 in the valve housing 16. The closing body 20 is held by means of a diaphragm 22, especially by means of a diaphragm 22 laterally adjoining this closing body 20 along a circumferential line of the closing body 20, and the closing body 20 together with the diagram 22 encloses a volume which is designated below as valve chamber 24. A gas, for example, ambient air, is pumped into this valve chamber 24 or pumped out of the valve chamber 24 by means of the valve drive 18. The quantity of gas in the interior of the valve chamber 24 determines the position of the closing body 20 and accordingly determines whether the valve 12, 14 is opened or closed or partially open or partially closed. The valve is closed if the closing body 20 is in contact with an edge of an end of a line section reaching into the valve housing 16, which edge is called a crater 26.

Each valve 12, 14 has a closing body 20, a diaphragm 22 holding the closing body 20 as well as a valve chamber 24 formed with the closing body 20 and the diaphragm 22, and an end of a line section closable by means of the closing body 20 protrudes into each valve housing 16, the end of which line section is designated as crater 26. In the view of the two valves 12, 14, these parts are each designated only in one of the two valves 12, 14 for the sake of better clarity.

The valve housing 16 of the exhalation valve 12 is either, as shown, open to the surrounding area or a line section open to the surrounding area is connected to the exhalation valve 12. The inhalation valve 14 is connected to a pressure source 30, especially to a medium pressure source, for example, to a gas cylinder. The pressure source 30 yields, for example, a pressure of 500 mbar in case of a gas cylinder functioning as medium pressure source. An end (crater 26) of a line section leading directly or indirectly from the pressure source 30 into the valve housing 16 of the inhalation valve 14 can be closed by means of the closing body 20 of the inhalation valve 14. When the inhalation valve 14 opens, i.e., when the closing body 20 thereof releases the crater 26, gas reaches from the pressure source 30 into an air path 32 in the interior of the ventilator 10.

The air path 32 has the shape of a “Y” in a manner common in case of the ventilation of a patient and thus has three “ends.” The exhalation valve 12 is located at a first end. The inhalation valve 14 is located at a second end. A third end leads to the patient and there, for example, to a breathing mask 34 worn by the patient, to an endotracheal tube or the like (patient interface).

When the inhalation valve 14 is open and the exhalation valve 12 is closed, gas coming from the pressure source 30 reaches the patient via the air path 32 in the interior of the ventilator 10 (inhalation; inspiratory phase). When the inhalation valve 14 is closed and the exhalation valve is open, a pressure equalization occurs from the patient's lungs to the surrounding area (exhalation; expiratory phase).

This function of the ventilator 10 as well as the function of the valves 12, 14 are basically known. The special feature of the ventilator 10 proposed here is the valve drive 18 of the valves 12, 14.

Each valve drive 18 comprises a plurality of pumping devices, which are designated below briefly as piezo pumps 40, 42 and which may also be regarded as “micropumps.” Such piezo pumps 40, 42 and the use thereof as valve drive 18 are likewise basically known per se and, to avoid unnecessary repetitions in the description being submitted here, reference is made to the older applications of the applicant with the official file Nos. 10 2016 009 833.3 (application date: Aug. 15, 2016) as well as 10 2017 009 606.6 (application date: Feb. 18, 2016), which shall herewith be considered to be included with their full disclosure contents in the description submitted here. A miniaturized valve drive 18 and overall a miniaturized valve 12, 14 are obtained with such piezo pumps 40, 42. The valves 12, 14 with such a valve drive 18 therefore especially come into consideration for use in a mobile ventilator 10 of the type mentioned in the introduction.

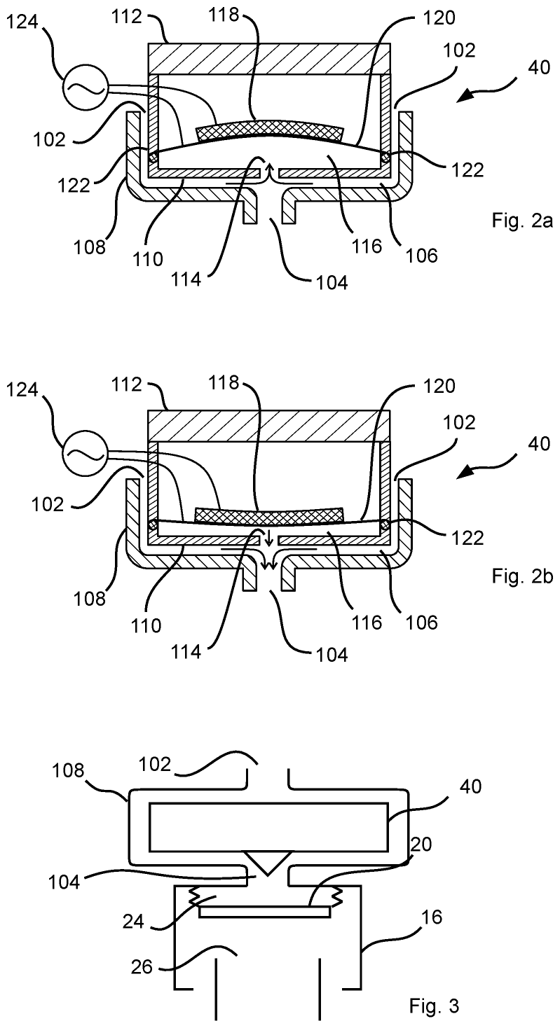

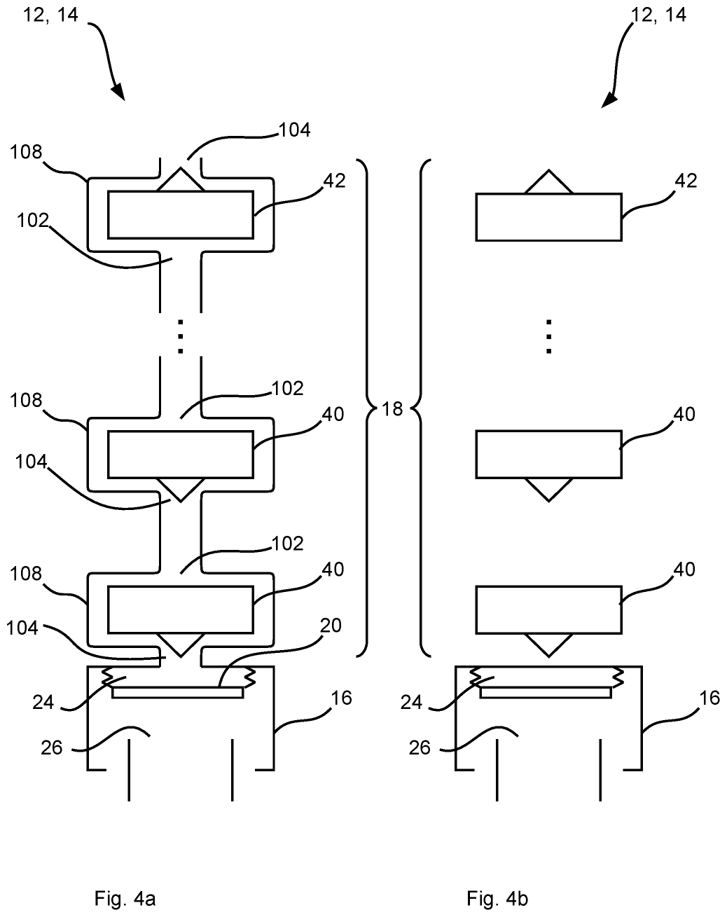

The views in FIG. 2 (FIGS. 2a, 2b) correspond to the views in FIGS. 4a and 4b of the last older application mentioned above.

FIG. 2 shows one of the piezo pumps (micropumps) according to FIG. 1 with additional details. According to this, a piezo pump 40 has a first two-way passage opening 102 and a second two-way passage opening 104, which are connected by a two-way duct 106. Because of the two-way duct 106, flow is possible through each piezo pump 40 and in two directions, namely, from the first two-way passage opening 102 to the second two-way passage opening 104, on the one hand, and from the second two-way passage opening 104 to the first two-way passage opening 102, on the other hand (bidirectional flow is possible).

The two-way duct 106 extends between an outer housing 108 and an inner housing 110 of the piezo pump 40. The second two-way passage opening 104 is formed in the outer housing 108. The first two-way passage opening 102 arises because of a space between an edge of the outer housing 108 and the adjoining inner housing 110. The inner housing 110 is closed by means of the cover plate 112.

A pumping opening 114, which connects the two-way duct 106 to a pump chamber 16, is arranged in the inner housing 110 in the two-way duct. A piezo element 118 and a pump diaphragm element 120 are arranged in the pump chamber 116. The pump diaphragm element 120 is connected to the piezo element 118, on the one hand, and to the inner housing 110, via flexible connection elements 122, on the other hand. Alternating electrical voltages are applied by means of an a.c. generator 124 to the piezo element 118 in a manner basically known per se. These alternating electrical voltages bring about a voltage-induced deformation of the piezo element 118 and this deformation leads to a controlled vibration of the pump diaphragm element 120. Because of a high-frequency a.c. voltage discharged by means of the a.c. generator 124, the pump diaphragm element 120 vibrates at a high frequency in the pump chamber 116 and pumping shocks are generated due to the resulting change in volume of the pump chamber 116 as a result (function of the piezo pump 40 as a high-frequency pump). These pumping shocks can work through the pumping opening 114 into the two-way duct 106 and bring about a flow of a respective fluid, for example, ambient air, through the second two-way passage opening 104.

The flow through the pumping opening 114, which is directed out of the pump chamber 116, is directed towards the second two-way passage opening 104. A pumping shock through the pumping opening 114, which is generated by a reduction in the volume of the pump chamber 116, is thus directed directly towards the second two-way passage opening 104. In this case, the flow between the pumping opening 114 and the second two-way passage opening 104 carries along the fluid in the two-way duct 106, so that a flow is generated from the first two-way passage opening 102 to the second two-way passage opening 104.

In the case of an increase in the volume of the pump chamber 116, the fluid is, by contrast, drawn out of the two-way duct 106 and through the pumping opening 114 into the pump chamber 116. The pumping opening 114 is arranged at a distance far from the second two-way passage opening 104 such that only a small percentage of fluid flows in the process through the second two-way passage opening 104 into the two-way duct 106 and finally through the pumping opening 114 into the pump chamber 116. The larger part of the fluid is drawn via the first two-way passage opening 102 into the two-way duct 106 and finally through the pumping opening 114 into the pump chamber 116. The volume drawn in this manner can again be discharged with a subsequent pumping shock in the direction towards the second two-way passage opening 104, and this brings about the above-described flow from the first two-way passage opening 102 to the second two-way passage opening 104.

By means of such pumping shocks, which are discharged from the piezo pump 40 at the frequency, with which the piezo element 118 is actuated by means of the a.c. generator 124, the valve chamber 24 is filled with the gas drawn by means of the piezo pump 40, especially ambient air, to move the closing body 20 (FIG. 1) of the valve 12, 14 (FIG. 1), in which the piezo pump 40 functions as part of the valve drive 18, so that a movement of the closing body 20 results. The closing body 20 can be moved so far due to a corresponding number of pumping shocks that this closing body 20 is pressed against the crater 26 of the line section leading to the valve housing 16. The number of pumping shocks and the gas volume thus being pumped into the valve chamber 24 also determines a pressure in the interior of the valve chamber 24 and thus a pressure, with which the crater 26 is kept closed by means of the closing body 20. The number of pumping shocks per time unit as well as the amplitude of the pumping shocks can be predefined by means of a corresponding actuation of the a.c. generator 124. Therefore, the speed of the movement of the closing body 20 (to the crater 26 or away from the crater 26), on the one hand, as well as the force (in defined limits in each case) acting on the closing boy 20 in the interior of the valve chamber 24, on the other hand, can be predefined by a respective actuation of the a.c. generator 124.

When mention is made here and below of a gas delivered or pumped by means of a piezo pump 40, the gas is preferably ambient air. Basically, any other flowable medium (fluid) instead of gas also comes into consideration.

No directed flow is present in the two-way duct 106 when the piezo pump 40 is switched off. There is then, rather, a free flow path through the two-way duct 106 between the first two-way passage opening 102 and the second two-way passage opening 104. A flow through the two-way duct 106 can be directed into both directions (bidirectional flow is possible). A pressure equalization can thus take place between the first two-way passage opening 102 and the second two-way passage opening 104. Hence, no relief valve or the like is needed.

The view in FIG. 3 illustrates the connection between the detailed view of a piezo pump 40 in FIG. 2 and the schematic view of the piezo pumps 40 in FIG. 1.

To this end, FIG. 3 shows a piezo pump 40 in the form, as it is symbolically shown in FIG. 1, but here additionally, even though this is insignificant in respect to the function, with a width “matching” the view of the piezo pump 40 in FIG. 2. The symbolic view obviously comprises a rectangle and a triangle adjoining the rectangle with its base. The rectangle represents the piezo pump 40 with the details explained in FIG. 2. The triangle represents the direction of action of the piezo pump 40 and points in the direction of the second two-way passage opening 104 of the piezo pump 40. The triangle thus to some extent symbolizes a direction of the “output” of the piezo pump 40. In the situation shown in FIG. 3, the triangle points in the direction of the valve chamber 24 of a valve housing 16. This means that the volume flow generated by means of the piezo pump 40 is directed towards the valve chamber 24 and that the resulting volume flow or at least a part of the resulting volume flow reaches the valve chamber 24 during the operation of the piezo pump 40. To this end, the piezo pump 40 with its outer housing 108 directly or indirectly adjoins the valve housing 16 in a suitable manner, so that a defined flow path arises from the output of the piezo pump 40 (the second two-way passage opening 104) to the valve chamber 24. For an illustration of this connection, the outer housing 108 of the piezo pump 40 is shown as an example in the view in FIG. 3 in a manner adjoining the pump housing 16 and adjoining the valve chamber 24 there (in the view in FIG. 1, the rectangle also comprises the outer housing 108 not shown there). In the view in FIG. 3, the outer housing 108 is, in addition, shown in a manner, in which the outer housing 108 in the area of the first two-way passage opening 102 allows a connection of an additional piezo pump 40, namely a connection of an outer housing 108 of an additional piezo pump 40.

On the basis of the view in FIG. 3 with the symbolic view of a piezo pump 40 explained there, the view in FIG. 4a shows a valve device 12, 14, which is considered to be an exhalation valve 12 or an inhalation valve 14, and with a plurality of piezo pumps 40, 42 comprised by the valve drive 18, as this was already shown in FIG. 1. The piezo pumps 40, 42 comprised by the valve drive 18 are connected to one another in a fluid-communicating manner by means of their outer housing 108 (i.e., each connected to one another in the area of the two-way passage openings 102, 104), wherein the connection between the outer housing 108 and an adjoining outer housing 108 in the valve drive 18 can be established between the outer housing 108 of a piezo pump 40, 42 and the outer housing 108 of an adjoining piezo pump 40, 42 in the valve drive 18, for example, also by means of at least one line section.

The view in FIG. 4b shows, in addition, the same valve device 12, 14 without the outer housing 108 of the piezo pumps 40 and without any line sections between the individual piezo pumps 40, 42 following one another in the valve drive 18. The view shown in FIG. 4b comprises both the same number of piezo pumps 40, 42 as the view shown in FIG. 4a and piezo pumps 40, 42 in the same direction of action as in the view shown in FIG. 4a. The view of the valve drive 18 in FIG. 4b corresponds essentially to the view of the valve drive 18 shown in FIG. 1. Thus, the connection is established between the (schematically highly simplified) view in FIG. 1, where the piezo pumps 40, 42 are likewise shown only symbolically in the form of a rectangle and of a triangle adjoining the rectangle with its base and without the outer housing 108 and any connection elements in the form of line sections or the like, and the detailed view of the valve drive 18 in FIG. 4a as well as the detailed view of the piezo pumps 40, 42 in FIG. 2.

The view in FIG. 4 (FIGS. 4a, 4b) shows a situation, in which the valve drive 18 comprises, by way of example, three or more piezo pumps 40, 42. The direction of action of the piezo pumps 40, 42 illustrated by means of the triangle in the symbolic view of the piezo pumps 40, 42 is directed towards the valve chamber 24 in case of at least two piezo pumps 40. The direction of action is reversed in case of at least one piezo pump 42. The direction of action thereof is thus directed away from the valve chamber 24. This piezo pump 42 with “reversed direction of action” is designated as inverse piezo pump 42 for distinction, wherein “inverse” refers only to the direction of action. The previous description of the function of a piezo pump 40 applies just as well to an inverse piezo pump 42, because the difference is only in the direction of action, i.e., in the “direction of installation” to some extent. A piezo pump 40 with a direction of action towards the valve chamber 24 is called a regular piezo pump 40 for distinction from an inverse piezo pump 42.

The indication of the basic similarity of a regular piezo pump 40 and of an inverse piezo pump 42 refers only to the functionality thereof and not to the dimensions or the like. The same generally applies to all piezo pumps 40, 42 comprised by the valve drive 18. All piezo pumps 40, 42 of a valve drive 18 may each have the same dimensions. This is, however, not a requirement, and the individual piezo pumps 40, 42 may have larger dimensions than others. This also includes possible differences in the increase in voltage and/or in the frequency range of the a.c. generator 124.

The piezo pumps 40, 42 form a line within the valve drive 18. This is possible since bidirectional flow is possible through each individual piezo pump 40, 42. Thus, bidirectional flow through the entire line is also possible. Accordingly, the position of the inverse piezo pump 42 along the line does not matter. The inverse piezo pump 42 may be located, as shown, “at the end” of the line (within the line located at the greatest distance from the valve chamber 24), “at the beginning” of the line or within the line.

Instead of a line with a series arrangement of piezo pumps 40, 42, a parallel arrangement is possible just as well. The description is continued on the basis of the views using the example of a series arrangement. A parallel arrangement can always be inferred.

The valve drive 18 may comprise one, two, three, four, five or more regular piezo pumps 40 as well as one, two or more inverse piezo pumps 42. The number of regular piezo pumps 40 comprised by the valve drive 18 is preferably greater than the number of inverse piezo pumps 42. In case of the embodiment shown, the valve drive 18 comprises exactly one inverse piezo pump 42 and a plurality of regular piezo pumps 40. This configuration is designated as “n+1 configuration,” and it shall thus be expressed that the valve drive 18 comprises basically any desired number of regular piezo pumps 40 and at least one inverse piezo pump 42.

The action and hence also the function of the inverse piezo pumps 42 in the valve drive 18 can be described briefly as follows: The regular piezo pumps 40 oriented with their direction of action towards the valve chamber 24 bring about a volume flow in the direction towards the valve chamber 24 in the activated state and deliver gas to the valve chamber 24 and at least partially into the valve chamber 24 in the activated state. The direction of action of the inverse piezo pump 42 and the volume flow resulting in case of the activation thereof (possibly a partial volume flow in case of the simultaneous activation of at least one regular piezo pump 40) is exactly reversed. An inverse piezo pump 42 in the activated state delivers gas away from the valve chamber 24 and as a result at least partially it delivers gas out of the valve chamber 24 as well. An inverse piezo pump 42 thus pumps gas out of the valve chamber 24 and as a result brings about a reduction in the volume of the valve chamber 24. By contrast, the regular piezo pump 40 or each regular piezo pump 40 pumps gas into the valve chamber 24 and as a result brings about an increase in the volume of the valve chamber 24. The reduction or increase in the volume of the valve chamber 24 leads to a corresponding displacement of the closing body 20.

The use of at least one additional piezo pump 42 (inverse piezo pump 42), through which flow is possible and which is oriented inversely compared to the other, regular piezo pumps 40 of the valve drive 18 hence makes possible an extended settability of the pressure acting on the closing body 20 on the valve chamber side. The pressure on the closing body 20 on the valve chamber side can be increased by means of one or a plurality of regular piezo pumps 40 (for closing the valve 12, 14 and for keeping the valve 12, 14 closed). The pressure on the closing body 20 on the valve chamber side can be reduced by means of one or a plurality of inverse piezo pumps 42. The reduction in the pressure on the closing body 20 can go into the negative range, so that the closing body 20 can be actively detached at least from the crater 26 in the interior of the valve housing 16 by means of an inverse piezo pump 42. This active detachment of the closing body 20 from the crater 26 is possible even without a counterpressure acting on the closing body 20 in the air path 32 in the interior of the ventilator 10. This means that the closing body 20 can be actively displaced into positions (for example, for a maximum opening of the valve 12, 14), for which a flow through the valve 12, 14 would otherwise be necessary.

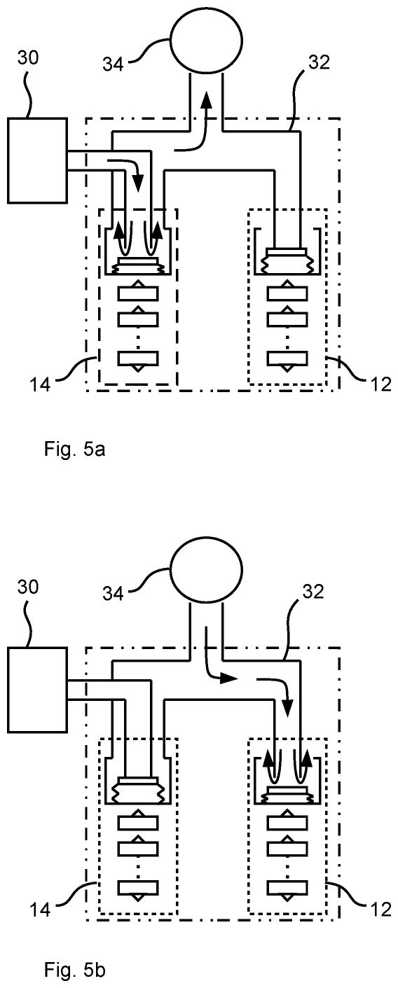

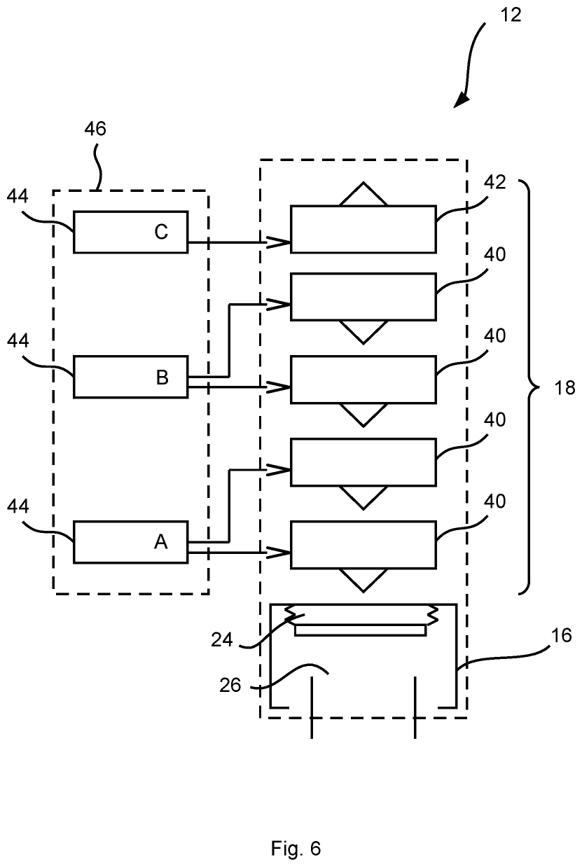

On the basis of the view in FIG. 1 and without all reference numbers from FIG. 1, the view in FIG. 5 (FIGS. 5a, 5b) now shows the gas flow in the air path 32 in the interior of the ventilator 10, especially from the pressure source 30 via the open inhalation valve 14 towards the patient, for example, to a breathing mask 34 worn by the patient, on the one hand (FIG. 5a), and from the patient via the open exhalation valve 12 to the surrounding area or to a pressure sink, on the other hand (FIG. 5b).

The view in FIG. 5a shows a snapshot of the positions of the valves 12, 14 during the inspiratory phase (inhalation valve 14 open; exhalation valve 12 closed). The view in FIG. 5b shows a snapshot of the positions of the valves 12, 14 during the expiratory phase (exhalation valve 12 open; inhalation valve 14 closed). The positions of the closing body 20 of the valves 12, 14 arising in the process are each set by means of the respective valve drives 18 (FIG. 1), i.e., each with at least one piezo pump 40 (FIG. 1, FIG. 2).

The special feature of the valve drive 18 of the ventilator 10 proposed here is that the valve drive 18—the valve drive 18 of the exhalation valve 12 or each exhalation valve 12 and/or the valve drive 18 of the inhalation valve 14 or of each inhalation valve 14—comprises a plurality of piezo pumps 40, 42 and has at least one inverse piezo pump 42 among them, as this is shown as an example in FIG. 1 and FIG. 4.

At the beginning of each expiratory phase, the exhalation valve 12 is open when the patient exhales. Hitherto, i.e., for example, in the case of a valve drive 18 with exactly one regular piezo pump 40 or with a plurality of such piezo pumps 40, the exhalation valve 12 is opened “passively” because of the difference in pressure between the patient's lungs and the surrounding area. The pressure in the patient's lungs is increased compared to ambient pressure because of the previous inspiratory phase. When the valve drive 18 of the exhalation valve 12 is deactivated, the resulting pressure difference is sufficient to open the exhalation valve 12, i.e., to detach the closing body 20 thereof from the crater 26 of the air path 32 ending in the valve housing 16 of the exhalation valve 12 in the interior of the ventilator 10. Because of the deactivated valve drive 18 and thus without the action of force on the valve chamber side (control side) on the closing body 20 and against the lung pressure, it is possible to speak of a passive opening of the exhalation valve 12 in case of such an opening of the exhalation valve 12.

This passive opening of the exhalation valve 12 is found to be unpleasant by the patient sometimes and requires that the patient exhale with a corresponding force against the exhalation valve 12. A few millibar may be necessary to open the exhalation valve depending on the volume enclosed by the valve chamber 24, a stroke of the closing body 20 and an elasticity of the border (diaphragm 22) of the closing body 20.

Active opening of the exhalation valve 12 is possible by means of at least one inverse piezo pump 42 in the valve drive 18 of the exhalation valve 12. An actively opened exhalation valve 12, and more precisely a flow resistance of an actively opened exhalation valve 12, is perceived either not at all or in any case markedly less than a passively opening exhalation valve 12 by a patient during the exhalation.

In the static state (FIG. 1), as long as no other forces are acting, the distance between the closing body 20 and the crater edge 26 is determined by the flexible diaphragm, which functions as a suspension of the closing body 20. If a volume flow (flow) to be controlled is led through the crater 26 in the direction toward the closing body 20, then this volume flow presses the closing body 20 away from the crater 26 by means of a building-up pressure in order to achieve a greater opening. The resulting pressure is designated as dynamic pressure for distinction from the pressure on the valve chamber side, which is also called control pressure. The dynamic pressure is built up (counterforce) against a force necessary for the deformation of the diaphragm 22. This counterforce on the valve chamber side and a resulting counterpressure are perceived as flow resistance of the exhalation valve 12 during the exhalation.

The exhalation valve 12 shall be closed (FIG. 5a) and the closing body 20 thereof shall close the crater 26 with a certain pressure/with a certain force during the inspiratory phase. At the beginning of the expiratory phase, the exhalation valve 12 is open (FIG. 5b) and an especially low flow resistance of the exhalation valve 12 is desirable precisely in the first moments of the exhalation. An especially low flow resistance of the exhalation valve 12 leads to the patient being able to exhale a certain volume especially easily in these first moments of the expiratory phase.

It was explained farther above that the pressure on the valve chamber side on the closing body 20 can be reduced by means of one or more inverse piezo pumps 42. This means that the pressure on the control side of the closing body 20 can be relieved not only passively (due to the exhalation of the patient), but also actively (by activation of the at least one inverse piezo pump 42). As a result, the closing body 20 can be actively moved into positions, for which a flow (for example, resulting during exhalation and) directed against the closing body 20 through the exhalation valve 12 would otherwise be necessary. This active opening of the exhalation valve 12 brings about a marked reduction in the flow resistance of the exhalation valve 12.

The view in FIG. 6 now shows, based on the view in FIG. 4b, a valve device functioning as an exhalation valve 12 with exactly five piezo pumps 40, 42, namely four regular piezo pumps 40 and an inverse piezo pump 42. This is an n+1 configuration as described above. Even if a configuration with exactly five piezo pumps 40, 42 (4+1) is shown here, other configurations with more or fewer regular piezo pumps 40 and/or more inverse piezo pumps 42 also come into consideration.

Control units 44 are provided and shown in a schematically simplified manner to actuate the piezo pumps 40, 42. The control units 44 are designated symbolically with the letters “A,” “B” and “C” for distinction and for easier referencing. In the situation shown, a control unit 44 each actuates two regular piezo pumps 40 (control unit “A,” control unit “B”). An additional control unit 44 (control unit “C”) actuates the inverse piezo pump 42. The control units 44 may be combined spatially and functionally into one control device 46.

A control signal of the control unit 44, which control signal is shown in the view in FIG. 6 by means of an arrow pointing to a piezo pump 40, 42 starting from the control unit 44, is an output signal of a signal generator comprised by the respective control unit 44, and in particular of a signal generator in the form of an a.c. generator 124 (FIG. 2). The control signal acts on the respective piezo element 118 of the piezo pump 40, 42. The two control units 44 each provided for actuation of two piezo pumps 40 (not shown) comprise each two signal generators independent of one another for the independent actuation of the two piezo pumps 40. Basically, an actuation of a plurality of piezo pumps 40 by means of a control unit 44 and exactly one signal generator comprised thereby is also possible. However, the piezo elements 118 of the piezo pumps 40 assigned to the control unit 44 cannot then be actuated independently of one another, i.e., be actuated with different frequencies and/or different amplitudes.

A pressure of 25 mbar, for example, can be applied by means of each piezo pump 40, 42. Because of the line-like combination (series arrangement) of the piezo pumps 40, 42 within the valve drive 18, the respective pressures generated are added up and the resulting sum acts in the valve chamber 24 and on the closing body 20 (such an addition also arises in case of a parallel arrangement). A pressure applied by means of a regular piezo pump 40 acts in a pressure-increasing manner. A pressure applied by means of the inverse piezo pump 42 acts in a pressure-lowering manner. When each of the piezo pumps 40, 42 can generate a pressure symbolically designated by p, the control pressure in the valve chamber 24 can be set in a range of −p up to ambient pressure and up to +4p ([−p . . . 4p]) by means of a combination of four regular piezo pumps 40 as well as one inverse piezo pump 42. A control pressure range of −25 mbar to +100 mbar correspondingly results in case of a pressure of p=25 mbar that can be applied by means of each piezo pump 40, 42.

The control pressure range resulting because of at least one inverse piezo pump 42 comprised by the valve drive 18 may be used

to press the closing body 20 actively against the crater 26 (the valve drive 18 generates a high positive control pressure; the valve 12, 14 closes),

to passively open the valve 12, 14 (all piezo pumps 40, 42 of the valve drive 18 are deactivated; the valve drive 18 generates no control pressure; the position of the closing body 20 is obtained because of the resting position of the diaphragm 22), or

to actively pull the closing body 20 away from the crater 26 (the valve drive 18 generates a negative control pressure; the valve 12, 14 opens beyond a position determined by the resting position of the diaphragm 22).

A “normal” flow resistance results in case of the passive opening of the valve 12, 14 (valve drive 18 deactivated). A flow resistance that is reduced compared to the “normal” flow resistance results in case of the active opening of the valve 12, 14 (valve drive 18 generates negative control pressure).

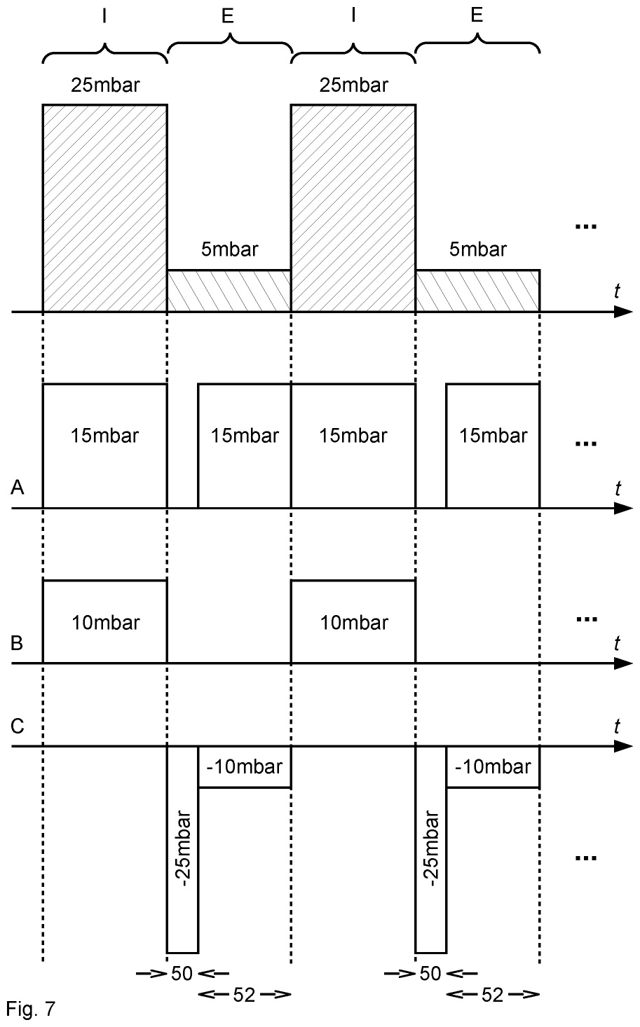

The view in FIG. 7 shows a utilization of the additive combination of the pressures generated by means of the piezo pumps 40, 42 comprised by the valve drive 18 during two consecutive breathing cycles each with an inspiratory phase and a subsequent expiratory phase. Individual pressure values are shown in an exemplary manner as columns each over the time t from top to bottom.

Desired values for the control pressure (pressure on valve chamber side) of the exhalation valve 12, which control pressure can be influenced by means of the valve drive 18, are shown at the very top. During the inhalation/inspiratory phase (designated symbolically by the capital letter “I”), the exhalation valve 12 shall be closed and is “kept closed” with a control pressure in the amount of 25 mbar. During the exhalation/expiratory phase (designated symbolically by the capital letter “E”), the exhalation valve 12 shall be at least partially open, but shall not be entirely open to obtain a so-called positive end expiratory pressure (PEEP), so that a control pressure of 5 mbar is provided.

In the next three sections of the view in FIG. 7, it is shown how this control pressure is generated by actuation of the piezo pumps 40, 42 comprised by the valve drive 18. For this purpose, the three additional timelines corresponding to the symbolic designation of the three control units 44 are designated by the capital letters “A,” “B” and “C” in FIG. 6. The pressure values shown over the timeline designated by “A” are thus desired values for the piezo pumps 40 actuated by means of the control unit 44 symbolically designated by “A.” The pressure values shown over the timeline designated by “B” are correspondingly desired values for the piezo pumps 40 actuated by means of the control unit 44 designated symbolically by “B” and the pressure values shown over the timeline designated by “C” are desired values for the inverse piezo pump 42 actuated by means of the control unit 44 designated symbolically by “C.”

By the two regular piezo pumps 40 assigned to the control unit 44 symbolically designated by “A” as well as the two regular piezo pumps 40 assigned to the control unit 44 symbolically designated by “B” generating each pressures of 15 mbar and 10 mbar, respectively, during an inspiratory phase (“I”), a total control pressure of 25 mbar arises in the valve chamber 24. A lower control pressure of 5 mbar is needed during the expiratory phase (“E”). In this case, the end of the expiratory phase is first considered in the view. The two regular piezo pumps 40 assigned to the control unit 44 that is symbolically designated by “A” are actuated there for generation of a pressure in the amount of 15 mbar. Furthermore, the inverse piezo pump 42 is actuated by means of the control unit 44 that is symbolically designated by “C.” A negative pressure, namely a pressure of −10 mbar, is generated by means of the inverse piezo pump 42 because of this actuation. In sum, a desired control pressure of 5 mbar corresponding to the desired value is obtained in the valve chamber 24. At the beginning of the expiratory phase, by contrast, the actually provided desired value of 5 mbar is intentionally fallen below to obtain an as low as possible flow resistance of the exhalation valve 12. FIG. 7 shows this in the form of an intermittent actuation, especially a maximum actuation (to obtain the maximum pressure that can be applied by the inverse piezo pump 42), only of the inverse piezo pump 42 by means of the control unit 44 symbolically designated by “C” to obtain a pressure of −25 mbar.

In regard to the actuation of the piezo pumps 40, 42 comprised by the valve drive 18, the expiratory phase is accordingly divided into an initial section 50 and a final section 52. An active opening of the exhalation valve 12 due to a corresponding actuation of the at least one inverse piezo pump 42 comprised by the valve drive 18 takes place at least during the initial section 50, which is also called the active phase 50 below, to obtain an as low as possible flow resistance of the exhalation valve 12.

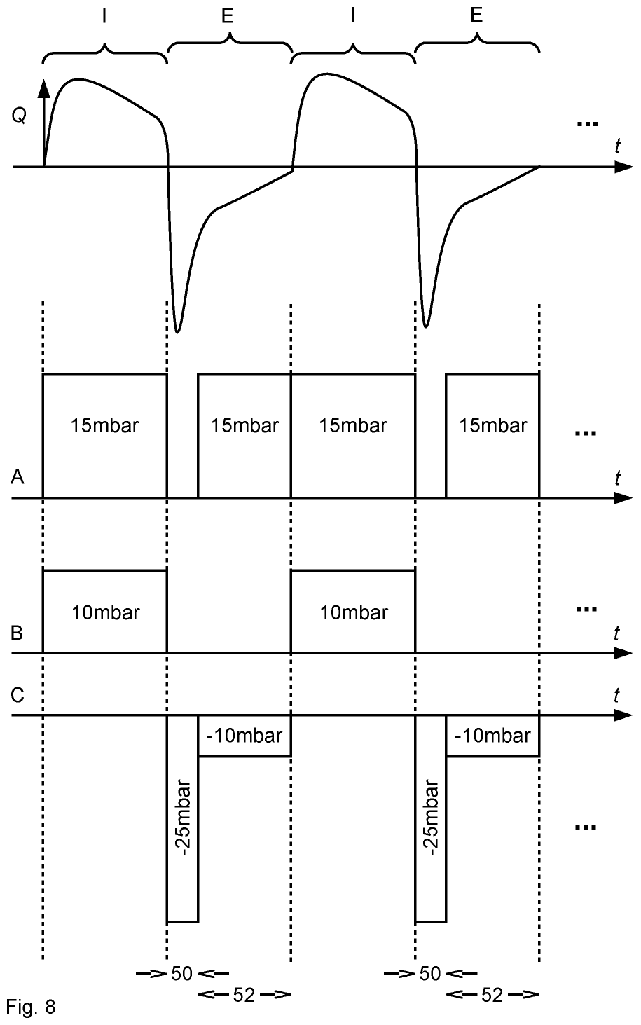

The resulting volume flow Q is shown for this purpose in the view in FIG. 8 via the just explained individual pressures generated by means of the piezo pumps 40, 42 of the valve drive 18 of the exhalation valve 12. As can be seen, a high negative volume flow Q out of the patient's lungs results during the active phase 50 (during the initial section 50) because of the active opening of the exhalation valve 12 at the beginning of the expiratory phase, so that, as a result, an especially simple exhalation process arises for the patient.

It can be seen in the views in FIG. 7 and FIG. 8 that the active opening of the exhalation valve 12 is only present at the start of the expiratory phase, namely during the active phase 50 (during the initial section 50 of the expiratory phase). The duration of the active phase 50 is predefined, for example, as a fraction of the overall duration of the expiratory phase, but may also be dependent on a measured value, as described separately farther below.

The duration of the active phase 50 can optionally also be changed, for example, for a physician or sufficiently medically qualified staff member. The duration of the active phase 50 is then a variable parameter determining the operation of the ventilator 10. The respective duration of the active phase 50 determines along with the overall duration of the expiratory phase (overall duration minus duration of the active phase 50) the time period, during which the piezo pumps 40, 42 comprised by the valve drive 18 of the exhalation valve 12 are actuated at the end of the expiratory phase (and during the final section 52) to guarantee the positive end expiratory pressure.

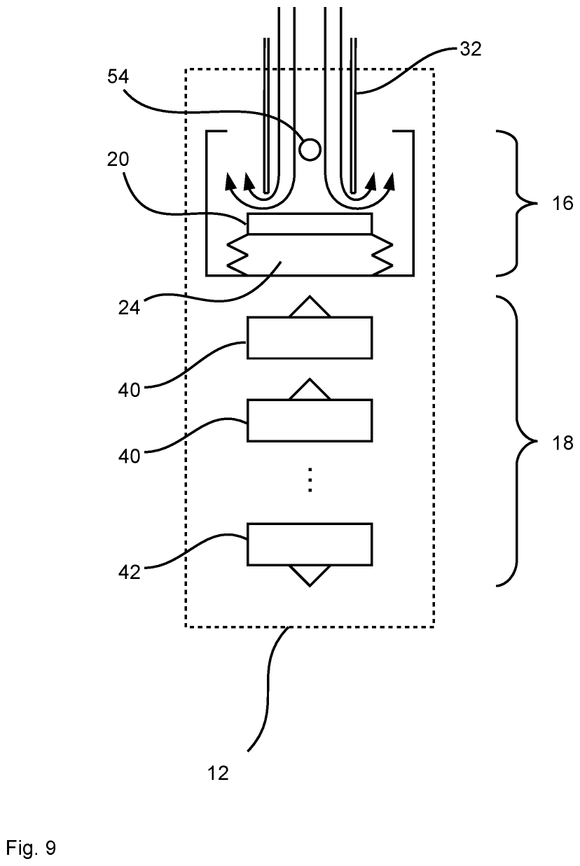

The curve of the airway pressure pAW, namely the curve of a corresponding measured value that can be obtained by the pressure sensor 54, can be monitored during the exhalation by means of a pressure sensor 54 assigned in space to the exhalation valve 12 (FIG. 9). As soon as the airway pressure pAW falls below a predefined or predefinable threshold value, for example, the pressure value provided as PEEP (here, for example, 5 mbar; see FIGS. 7, 8), a measured-value-specific changeover between the active phase 50 and the final section 52 can take place.

The view in FIG. 9 shows to this extent the exhalation valve 12 from FIG. 1 with a volume flow coming from the air path 32 in the interior of the ventilator 10 (i.e., breathing air exhaled by the patient) and with a pressure sensor 54 for detecting the airway pressure pAW. The pressure sensor 54 is assigned in space to the exhalation valve 12 (located in the exhalation valve 12 or close to the exhalation valve 12) and located in the direction of the volume flow in front of the exhalation valve 12, at least in front of the closing body 20 of the exhalation valve 12 (upstream of the exhalation valve 12 or upstream of the closing body 20 of the exhalation valve 12).

The measured value of the pressure sensor 54 may optionally be used not only for the automatic and sensor-controlled ending of the active phase 50 during the exhalation, but also, in addition or as an alternative, for control to a positive end expiratory pressure. For this, a difference from the airway pressure pAW as measured value of the pressure sensor 54 (actual value) and a desired value for the airway pressure, which desired value is valid during the final section 52 of the expiratory phase, is fed in a manner basically known per se to a controller, not shown, for example, to a P controller, to a PI controller or to a PID controller. The controller acts on the valve drive 18 of the exhalation valve 12, i.e., it generates a set value for the valve drive 18. As a result, the controller corrects the position of the closing body 20 as a function of a respective, actual airway pressure pAW, so that the desired value for the airway pressure, which desired value is valid during the final section 52 of the expiratory phase, is obtained as readily as possible. By contrast to a pure control of the position of the closing body 20 due to a corresponding actuation of the valve drive 18, for example, aging- and/or temperature-related influences especially on the diaphragm can hence be compensated.

The position of the closing body 20 of a valve 12, 14 is generally obtained based on the sum of all forces which act on the closing body 20. A dynamic force F_{S} based on an incoming volume flow, a restoring force F_{R} and weight forces F_{G} based on the mass of the closing body 20 and the mass of parts of the diaphragm 22 are added up. The dynamic force F_{S} (F_{S}=Δp×A_{S}) is obtained with the pressure difference Δp between a pressure p1 “in front of” the valve 12, 14 and pressure p2 “behind” the valve 12, 14 as well as with an area A_{S} of the closing body 20 exposed to the dynamic pressure. The weight force F_{G} may take different directions in case of different installation positions of the valve 12, 14 as they may arise, for example, in case of a transportable ventilator 10. The restoring force F_{R} is obtained especially from a force/path curve of the diaphragm 22. The restoring force F_{R} is aging- and temperature-dependent. Because of the direction of the action of the weight force F_{G}, which direction is dependent on the installation position, as well as the time- and temperature-dependent value of the restoring force F_{R}, a control of the pressure, which is generated by means of the piezo pumps 40, 42 comprised by the valve drive 18 and acts on the closing body 20 of the respective valve 12, 14, is meaningful in the manner outlined above for compensation of otherwise resulting errors in the positioning of the closing body 20. When only the position-dependent action of the weight force F_{G} is considered, the closing body 20 can be deflected in different directions in case of a deactivated valve drive 18 starting from the zero position thereof because of the weight force, to some extent into a “positive” direction starting from the zero position or into a “negative” direction starting from the zero position, and a static error arises for the position of the closing body 20. By the valve drive 18 comprising at least one inverse piezo pump 42, the valve drive 18 is able to compensate a static error in both directions in regard to the position of the closing body 20.

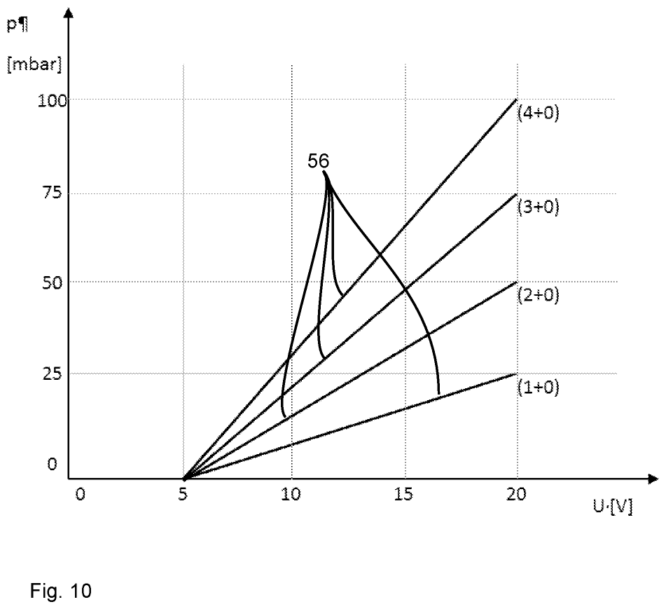

The use of at least one inverse piezo pump 42 within a valve drive 18 which comprises a plurality of piezo pumps 40, 42 also has, in addition, the advantage of a good settability of a characteristic 56 of the respective valve device 12, 14. For this, the view in FIG. 10 shows a family of characteristics with four characteristics 56, each characteristic 56 belonging to a valve drive 18 with a defined number of regular piezo pumps 40. The characteristics 56 thus belong to a valve drive 18 without an inverse piezo pump 42. The characteristics 56 belong to valve drives 18 with exactly one regular piezo pump 40, exactly two regular piezo pumps 40, exactly three regular piezo pumps and exactly four regular piezo pumps 40 and the individual characteristics 56 are correspondingly designated by “(1),” “(2),” “(3)” and “(4)” for distinction. The characteristics 56 are plotted on the abscissa over an operating voltage U in volts and on the ordinate over a pressure p in millibar, the amplitude of the signal outputted by the a.c. generator 124 being set as the operating voltage U. No noteworthy resulting pressure arises with an operating pressure below 5 V.

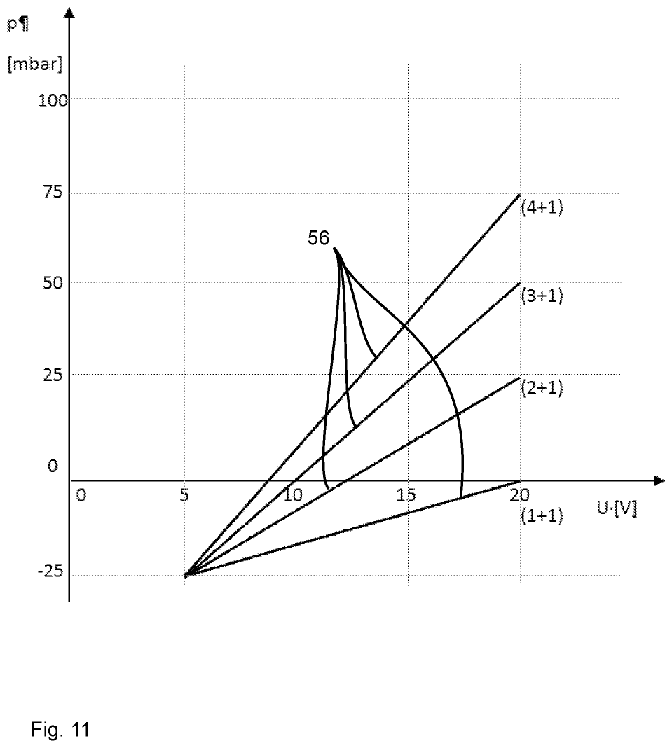

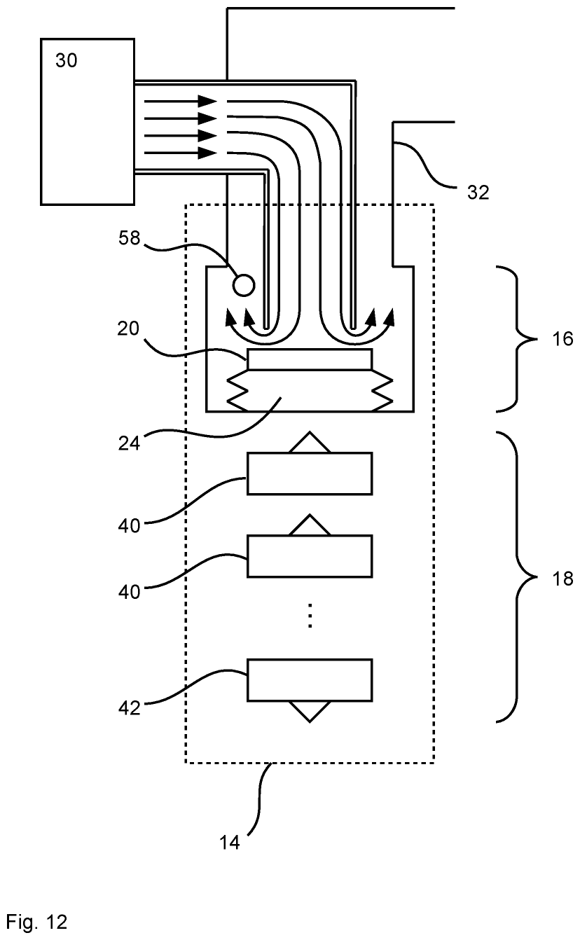

The view in FIG. 11 shows a family of characteristics with a plurality of characteristics 56 for a valve drive 18 with the same number of regular piezo pumps 40 as in FIG. 10 and additionally with an inverse piezo pump 42 each. As can be seen, a good setting of the resulting pressure p around the zero point is hence also now possible due to corresponding operating voltage U. This is advantageous because desired PEEP pressures mostly range in the lower millibar range between 0 mbar and 10 mbar. In case of a valve drive 18 without at least one inverse piezo pump 42, the result is that it is necessary to operate in a curved and highly sample-dependent as well as temperature-dependent range of the characteristic 56. Due to the use of at least one inverse piezo pump 42 in the valve drive 18, for example, the inverse piezo pump 42 and the regular piezo pump 40 or a plurality of regular inverse piezo pumps 40, 42 are each able to work in a medium range of their respective characteristic 56 for low required control pressures. The resulting control pressures then correspond to the difference of both actively operated piezo pumps 40, 42.

In summary, it can be determined that in case of a valve device 12, 14 functioning as an exhalation valve 12 with a valve drive 18 with a plurality of regular piezo pumps 40 and at least one inverse piezo pump 42 during the inspiratory phase, a closing of the valve 12 even against high inhalation pressure (up to 100 mbar) can be guaranteed by a sufficient plurality of regular piezo pumps 40 being activated. A transition from the inspiratory phase (closed, high counterpressure) into the expiratory phase (open, low resistance, settable counterpressure) shall be carried out rapidly. For this, the exhalation valve 12 is actively opened by at least brief activation (active phase 50) of the at least one inverse piezo pump 42. The change in the position of the closing body 20 (away from the crater 26) and as a result the active opening can take place very rapidly due to a, for example, maximum or approximately maximum activation of the at least one inverse piezo pump 42, wherein the speed of the opening is dependent on the pressure that can be applied to the at least one inverse piezo pump 42 and hence can be influenced by a corresponding actuation of the at least one inverse piezo pump 42. Subsequently (final section 52 of the expiratory phase), it shall be controlled to a settable low counterpressure (PEEP), wherein the at least one inverse piezo pump 42 and at least one regular piezo pump 40 are active for this in order to keep the joint operation of the piezo pumps 40, 42 in a favorable range of the respective characteristic 56.

The view in FIG. 12 shows a valve device 12, 14 functioning as inhalation valve 14 with a volume flow coming from the pressure source 30, which volume flow reaches the air path 32, shown only partially, in the interior of the ventilator 10 in case of an open inhalation valve 14 (FIG. 1). For a control of the position of the closing body 20 of a valve device 12, 14 functioning as an inhalation valve 14 with a valve drive 18 with a plurality of regular piezo pumps 40 and at least one inverse piezo pump 42, a pressure sensor 58 assigned in space to the inhalation valve 14 is located in the air path 32 in the interior of the ventilator 10 for a detection of a measured value of an airway pressure pAW.

The control of an inhalation valve 14 is carried out, in principle, just as this was already described farther above for the control of an exhalation valve 12. In case of the control of an inhalation valve 14 to a desired airway pressure during the inspiratory phase, a difference from the airway pressure pAW as measured value of the pressure sensor 58 (actual value) and an optionally time-dependent desired value for the airway pressure is fed in a manner basically known per se to a controller, not shown, for example, to a P controller, to a PI controller or to a PID controller. The controller acts on the valve drive 18 of the inhalation valve 14 and generates a set value for the valve drive 18. As a result, the controller corrects the position of the closing body 20 of the inhalation valve 14 as a function of the respective, actual airway pressure pAW, so that the desired value of the airway pressure that is valid during the inspiratory phase is obtained as readily as possible. By contrast to a pure control of the position of the closing body 20 due to a corresponding actuation of the valve drive 18, for example, aging- and/or temperature-dependent changes of the diaphragm 22 can also be compensated here as a result. In addition, a very accurate compliance with a desired curve of the airway pressure can be guaranteed by means of the control in case of a desired value for the airway pressure during the inspiratory phase, which desired value is time-dependent and variable during the inspiratory phase, for example, in case of a volume-controlled ventilation.

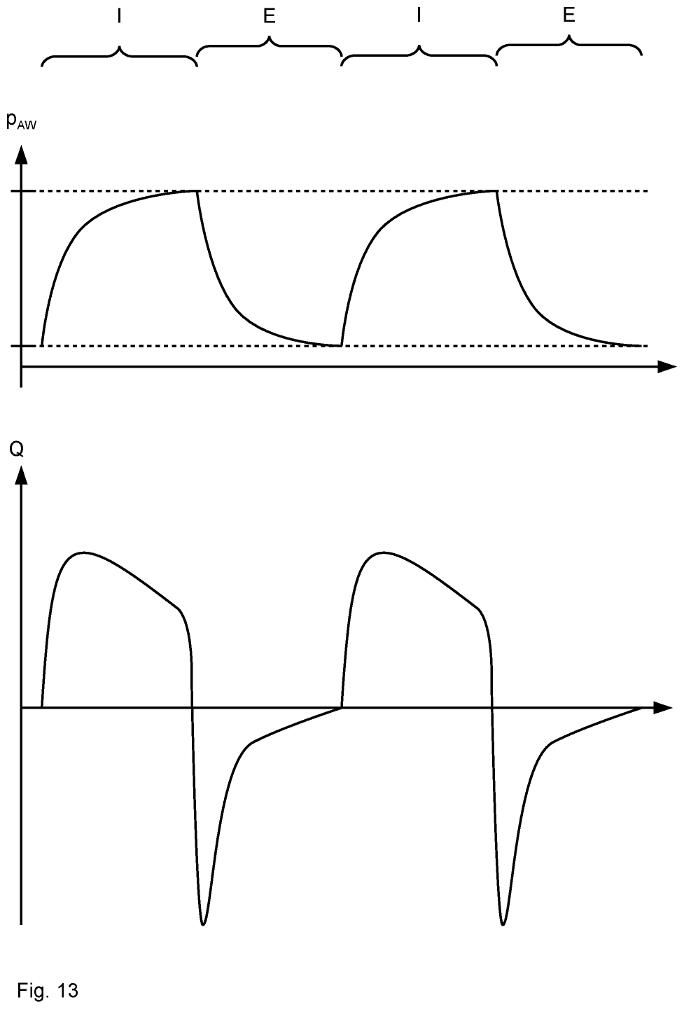

The view in FIG. 13 shows a plurality of ventilation cycles each with consecutive inspiratory phases (“I”) and expiratory phases (“E”) and for the individual phases each with the time curve of an airway pressure pAW (FIG. 13, top) as well as the time curve of the resulting volume flow Q (FIG. 13, bottom) from the ventilator 10 to the patient's lungs during the inspiratory phase and from the patient's lungs to the ventilator 10 and via the exhalation valve 12 out of the ventilator 10 during the expiratory phase. The airway pressure pAW fluctuates between a lower threshold value and an upper threshold value each illustrated with broken horizontal lines. The lower threshold value arises based on the respective provided positive end expiratory pressure (PEEP). The upper threshold value is the desired value for the airway pressure pAW during the inspiratory phase. The lower threshold value (PEEP) is, for example, 5 mbar. The upper threshold value is, for example, 25 mbar.

The airway pressure can be kept between the lower threshold value and the upper threshold value due to a controlled actuation of the valve drive 18 of the inhalation valve 14 and due to the same controlled actuation of the valve drive 18 of the exhalation valve 12 during consecutive inspiratory and expiratory phases. The volume flow Q rapidly increases sharply at first at the beginning of an expiratory phase (because of the large pressure difference between the pressure level of the pressure source 30 and the pressure in the air path 32 in the interior of the ventilator 10 immediately after the opening of the inhalation valve 14). With increasing pressure equalization and increasing approach to the desired value for the airway pressure pAW, the volume flow Q drops again starting from its maximum value and a high negative volume flow Q arises because of pressure equalization between the patient's lungs and the surrounding area in case of a changeover between an inspiratory phase and a subsequent expiratory phase in case of the active opening of the exhalation valve 12, and the active opening, as described, permits the especially high negative volume flow Q at the beginning of the expiratory phase and makes it easier for the patient to exhale.

Individual principal aspects of the description submitted here can hence be summarized briefly as follows: Proposed is a ventilator 10, which comprises at least one exhalation valve 12 and/or at least one inhalation valve 14 with a valve drive 18 intended for influencing a position of a closing body 20 of the respective valve 12, 14, wherein the valve drive 18 acts on a valve chamber 24 and a volume in the valve chamber 24 determines the position of the closing body 20, and wherein the valve drive 18 comprises a plurality of piezo pumps 40, namely at least one regular piezo pump 40 with a direction of action towards the valve chamber 24 as well as at least one inverse piezo pump 42 with a reversed direction of action. Proposed is likewise a process for operating such a ventilator 10, namely a process, in which the valve 12, 14 comprising at least one inverse piezo pump 42 is actively opened by means of this at least one inverse piezo pump. Overall, valves 12, 14, which can especially be used as exhalation valve or inspiration valve 12, 14, with a valve drive 18 with at least one inverse piezo pump 42 comprised by it for the active opening of the valve 12, 14 and a process for operating such a valve 12, 14 is proposed, wherein the at least one inverse piezo pump 42 is actuated for the active opening of the valve 12, 14 within the framework of the process.

While specific embodiments of the invention have been shown and described in detail to illustrate the application of the principles of the invention, it will be understood that the invention may be embodied otherwise without departing from such principles.

### LIST OF REFERENCE NUMBERS

10 Ventilator

12 Exhalation valve, valve, valve device

14 Inhalation valve, valve, valve device

16 Valve housing

18 Valve drive

20 Closing body

22 Diaphragm

24 Valve chamber

26 Crater

28 (blank)

30 Pressure source

32 Air path

34 Breathing mask

36, 38 (blank)

40 Piezo pump, regular piezo pump

42 Piezo pump, inverse piezo pump

44 Control unit

46 Control device

48 (blank)

50 Active phase, initial section of the expiratory phase

52 Final section of the expiratory phase

54 Pressure sensor

56 Characteristic

58 Pressure sensor

102 First two-way passage opening

104 Second two-way passage opening

106 Two-way duct

108 Outer housing

110 Inner housing

112 Cover plate

114 Pumping opening

116 Pump chamber

118 Piezo element

120 Pump diaphragm element

122 Connection element

124 A.c. generator

1. A ventilator comprising at least one exhalation and/or inhalation valve comprising:
a valve drive; and
a closing body, wherein
the valve drive is configured to influence a position of the closing body;
the valve drive is configured to act on a valve chamber;
a volume within the valve chamber determines the position of the closing body; and
the valve drive comprises a plurality of piezo pumps, including at least one piezo pump with a direction of action towards the valve chamber and at least one piezo pump with a direction of action away from the valve chamber.

2. A ventilator in accordance with claim 1, wherein the at least one piezo pump with a direction of action towards the valve chamber as well as the at least one piezo pump with a direction of action away from the valve chamber are in a series arrangement.

3. A ventilator in accordance with claim 1, wherein the at least one piezo pump with a direction of action towards the valve chamber as well as the at least one piezo pump with a direction of action away from the valve chamber are in a parallel arrangement.

4. A ventilator in accordance with claim 1, wherein the valve drive of a valve, functioning as exhalation valve or as inhalation valve comprises exactly one piezo pump with a direction of action away from the valve chamber and a plurality of piezo pumps with a direction of action towards the valve chamber.

5. A ventilator in accordance with claim 3, wherein a nonreturn valve is arranged in the valve drive at the at least one piezo pump with the direction of action towards the valve chamber such that no escape of the volume from the valve chamber against the direction of action is made possible.

6. A ventilator in accordance with claim 3, wherein a nonreturn valve is arranged in the valve drive at the at least one piezo pump with a direction of action away from the valve chamber such that no escape of the volume from the valve chamber against the direction of action is made possible.

7. A process for operating a ventilator, the process comprising the steps of:
providing the ventilator with a valve comprising a valve drive and a closing body, wherein the valve drive is configured to influence a position of the closing body, the valve drive is configured to act on a valve chamber, a volume within the valve chamber determines the position of the closing body and the valve comprises at least one piezo pump with a direction of action away from the valve chamber; and
actively opening the valve, by means of the at least one piezo pump with a direction of action away from the valve chamber.

8. A process in accordance with claim 7, further comprising the steps of:
providing a pressure sensor; and
detecting a measured pressure value with the pressure sensor assigned in space to a valve of the ventilator; and
regulating a position of the closing body of the valve by means of the measured pressure value as an actual value and by means of a predefined or predefinable pressure value as a desired value.

9. A process) in accordance with claim 7, wherein when the valve functions as an exhalation valve with the at least one piezo pump with a direction of action away from the valve chamber, the exhalation valve is actively opened by means of the at least one piezo pump with a direction of action away from the valve chamber.

10. A process in accordance with claim 9, wherein the exhalation valve is actively opened at the beginning of an expiratory phase.

11. A process in accordance with claim 10, wherein the exhalation valve is actively opened at the beginning of the expiratory phase for a predefined or predefinable duration.

12. A process in accordance with claim 11, wherein the exhalation valve is actively opened at the beginning of the expiratory phase and remains opened until a measured pressure value detected by means of a pressure sensor assigned in space to the exhalation valve falls below a predefined or predefinable threshold value.

13. A process according to claim 7, wherein a computer program with program code means carries out at least some of the steps when the control program is run on a control device for the ventilator.

14. A ventilator comprising:
a valve comprising:
a closing body and a valve chamber partially defined by the closing body, a volume within the valve chamber determining a position of the closing body; and
a valve drive configured to influence a position of the closing body by acting on the valve chamber, the valve drive comprising a plurality of piezo pumps, including at least one piezo pump with a direction of action towards the valve chamber and at least one piezo pump with a direction of action away from the valve chamber; and
a control unit configured to control the at least one piezo pump with a direction of action away from the valve chamber to actively open the valve.

15. A ventilator in accordance with claim 14, wherein the at least one piezo pump with a direction of action towards the valve chamber as well as the at least one piezo pump with a direction of action away from the valve chamber are in a series arrangement or in a parallel arrangement.

16. A ventilator in accordance with claim 14, wherein the valve drive, with the valve functioning as exhalation valve or as inhalation valve comprises exactly one piezo pump with a direction of action away from the valve chamber and a plurality of piezo pumps with a direction of action towards the valve chamber.

17. A ventilator in accordance with claim 16, further comprising:
a nonreturn valve arranged in the valve drive at the at least one piezo pump with the direction of action towards the valve chamber such that there is no escape of the volume from the valve chamber against the direction of action; or
a nonreturn valve is arranged in the valve drive at the at least one piezo pump with a direction of action away from the valve chamber such that there is no escape of the volume from the valve chamber against the direction of action; or
a nonreturn valve arranged in the valve drive at the at least one piezo pump with the direction of action towards the valve chamber such that there is no escape of the volume from the valve chamber against the direction of action and a nonreturn valve is arranged in the valve drive at the at least one piezo pump with a direction of action away from the valve chamber such that there is no escape of the volume from the valve chamber against the direction of action.

18. A ventilator in accordance with claim 14, further comprising a pressure sensor, wherein:
the pressure sensor detects a pressure value of the volume; and
the control unit is configured to control the at least one piezo pump with a direction of action away from the valve chamber by regulating a position of the closing body of the valve by means of the measured pressure value as an actual value and by means of a predefined or predefinable pressure value as a desired value.

19. A ventilator in accordance with claim 14, wherein the valve functions as an exhalation valve with the exhalation valve actively opened by means of the at least one piezo pump with a direction of action away from the valve chamber.

20. A ventilator in accordance with claim 14, wherein the exhalation valve is actively opened at the beginning of an expiratory phase.
